(19) United States
(12) Patent Application Publication
(10) Pub. No.: US 2005/0142595 A1
(43) Pub. Date: Jun. 30, 2005

(54) Intercalator FRET donors or acceptors

(75) Inventors: Anthony M. Maletta, Woburn, MA (US); Eric A. Nalefski, Reading, MA (US)

Correspondence Address: WOLF GREENFIELD & SACKS, PC;FEDERAL RESERVE PLAZA, 600 ATLANTIC AVENUE, BOSTON, MA, 02210-2211, US

(73) Assignee: U.S. Genomics, Inc., Woburn, MA (US)

(21) Appl. No.: 10/984,191

(22) Filed: Nov. 8, 2004

Related U.S. Application Data
(60) Provisional application No. 60/518,485, filed on Nov. 7, 2003.

(51) Int. Cl. C12Q 1/68; C07H 21/04

(52) U.S. Cl. 435/6; 536/24.3

(57) ABSTRACT

The invention relates to methods and products for analyzing nucleic acids using FRET. In particular the methods involve improvements in FRET signaling and in some instances utilize intercalators as part of a fluorophore pair.

### RELATED APPLICATIONS

This application claims priority under 35 U.S.C. § 119(e) to U.S. Provisional Application Ser. No. 60/518,485, entitled “Intercalator FRET Donors or Acceptors,” filed on Nov. 7, 2003, which is herein incorporated by reference in its entirety.

### FIELD OF THE INVENTION

The present invention relates generally to FRET based methods and related compositions for nucleic acid analysis.

### BACKGROUND OF THE INVENTION

The study of molecular and cellular biology is focused on the microscopic structure of cells. It is known that cells have a complex microstructure that determines the functionality of the cell. Much of the diversity associated with cellular structure and function is due to the ability of a cell to assemble various building blocks into diverse chemical compounds. The cell accomplishes this task by assembling nucleic acids from a limited set of building blocks referred to as monomers. One key to the diverse functionality of nucleic acids is based in the primary sequence of the monomers within the nucleic acid. This sequence is integral to understanding the basis for cellular function, such as why a cell differentiates in a particular manner or how a cell will respond to treatment with a particular drug.

The ability to identify the structure of nucleic acids by identifying the sequence of monomers is integral to the understanding of each active component and the role that component plays within a cell. By determining the sequences of nucleic acids it is possible to generate expression maps, to determine what proteins are expressed, to understand where mutations occur in a disease state, and to determine whether a nucleic acid has better function or loses function when a particular monomer is absent or mutated.

Many technologies relating to genomic sequencing and analysis require site-specific labeling of nucleic acids. Most site-specific labeling is carried out using nucleic acid based probes that hybridize to their complementary sequences within a nucleic acid target. The specificity of these probes will vary however depending upon their length, their sequence, the hybridization conditions, and the like. The ability to increase the specificity of these probes and, at the same time, use less of them would make labeling reactions more efficient and less expensive to run.

### SUMMARY OF THE INVENTION

The invention relates to methods and related compositions for nucleic acid analysis using an improved fluorescence resonance energy transfer (FRET) based analysis. In one aspect the invention is a method for analyzing a nucleic acid by contacting a nucleic acid with an intercalator fluorophore and a sequence specific probe capable of hybridizing to the nucleic acid, wherein the probe is labeled with a probe fluorophore, and detecting fluorescence or quenching arising from FRET between the intercalator fluorophore and the probe fluorophore to analyze the nucleic acid.

Optionally, the intercalator fluorophore is tethered to the same probe or to a second preferably sequence-specific probe which is capable of hybridizing to an adjacent section of the nucleic acid to the probe labeled with the probe fluorophore.

In another aspect the invention is a composition comprising a probe tethered to an intercalator fluorophore and a probe fluorophore, wherein the intercalator fluorophore and the probe fluorophore comprise a fluorophore pair.

Various embodiments appear equally to the different aspects of the invention. These are recited below.

In one embodiment the intercalator fluorophore is tethered to one end of the probe and the probe fluorophore is tethered to the other end of the probe.

In one embodiment the intercalator fluorophore is tethered to the probe. In another embodiment the intercalator fluorophore is separate from the probe. The intercalator fluorophore may be a donor or acceptor fluorophore. The probe fluorophore may be an acceptor or donor fluorophore.

The probe and/or intercalator fluorophore may be tethered directly to the probe. In other embodiments the probe fluorophore and/or the intercalator fluorophore is tethered to the probe through a linker. In yet other embodiments the probe fluorophore and/or the intercalator fluorophore is tethered to a terminal or an internal nucleotide of the probe.

The nucleic acid may be single stranded or double stranded.

Each of the limitations of the invention can encompass various embodiments of the invention. It is therefore anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways.

The phraseology and terminology used herein is for the purpose of description and should not be regarded as limiting. The use of “including”, “comprising”, or “having”, “containing”, “involving”, and variations thereof herein, is meant to encompass the items listed thereafter and equivalents thereof as well as additional items.

### BRIEF DESCRIPTION OF THE DRAWINGS

The figures are illustrative only and are not required for enablement of the invention disclosed herein.

FIG. 1 is a schematic diagram depicting some embodiments of the invention. Several nucleic acid strands are depicted schematically and labeled as random strand, target strand and probe strand with acceptor. D refers to an intercalator that functions as a donor. A refers to an acceptor. When the target strand hybridizes with the probe, the donor intercalator is incorporated into the double stranded region and is capable of FRET with the acceptor.

FIG. 2 is a schematic diagram depicting other embodiments of the invention. Several nucleic acid strands are depicted schematically and labeled as random strand, target strand and probe strand with acceptor and donor attached. D refers to an intercalator that functions as a donor. A refers to an acceptor. When the target strand hybridizes with the probe, the donor intercalator is incorporated into the double stranded region and is capable of FRET with the acceptor.

### DETAILED DESCRIPTION OF THE INVENTION

Methods and related compositions for identifying information about a nucleic acid, such as the nucleotide sequence are described. In one aspect, the methods involve contacting a nucleic acid with an intercalator fluorophore and a sequence specific probe capable of hybridizing to the nucleic acid. The probe is labeled with a probe fluorophore. Fluorescence or quenching arising from FRET between the intercalator fluorophore and the probe fluorophore is detected to analyze the nucleic acid.

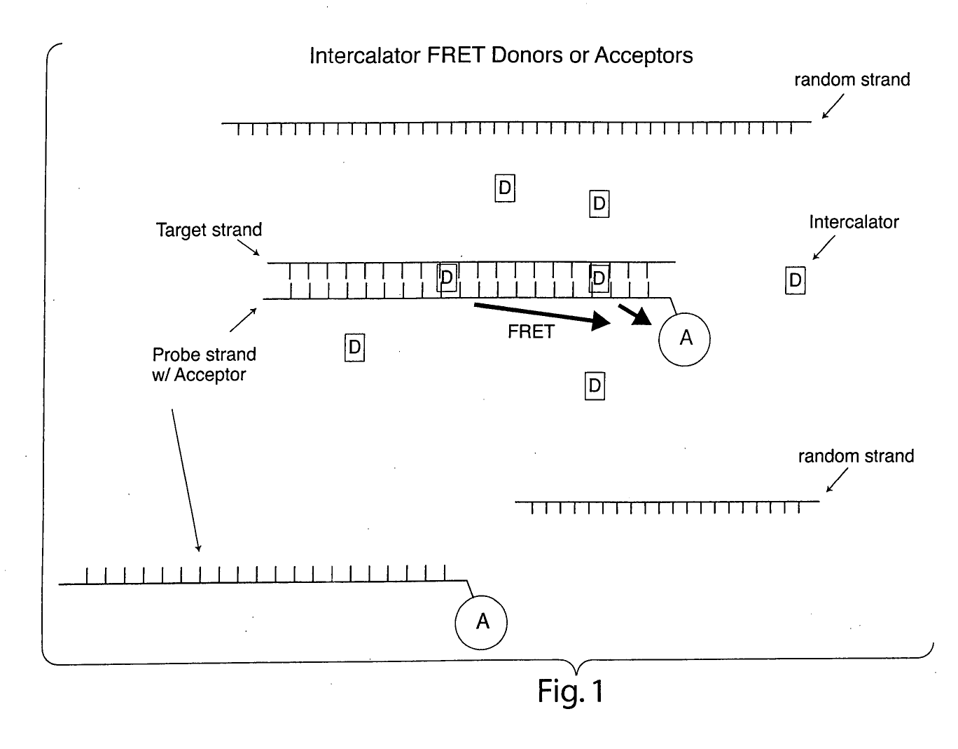

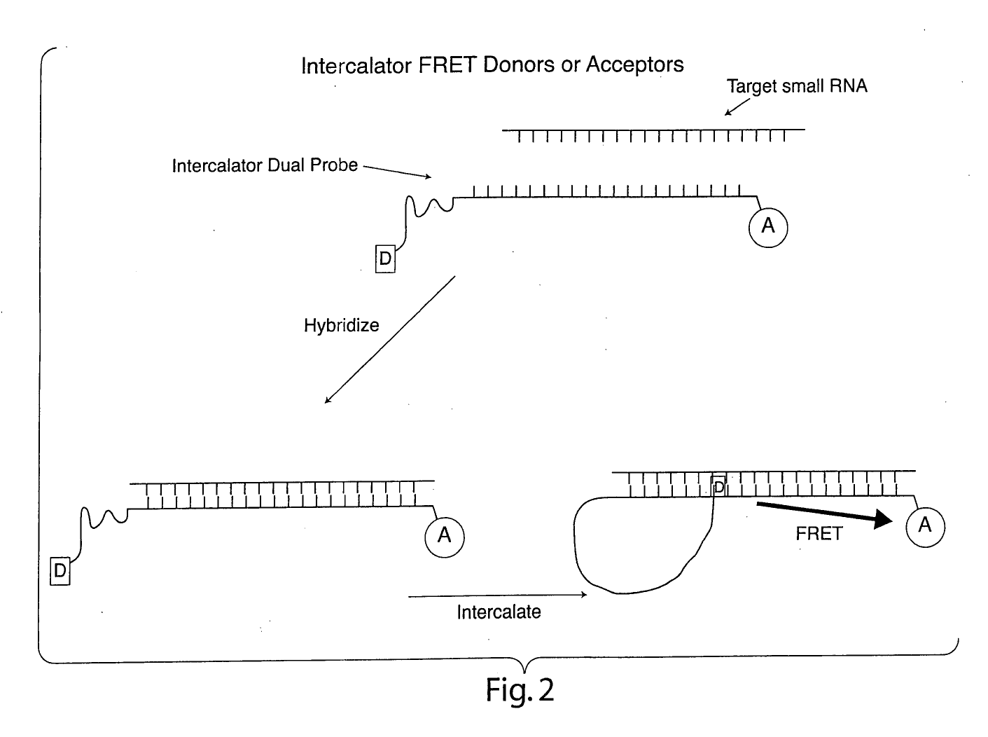

The intercalator fluorophore and the probe fluorophore are a fluorophore pair. When the members of the fluorophore pair are positioned in proximity to one another by hybridization of the probe to the nucleic acid, a signal is generated by FRET. This may be accomplished in several ways. Two exemplary methods for accomplishing this are depicted in FIGS. 1 and 2.

FIG. 1 is a schematic diagram of some examples of the methods of the invention. The exemplary method depicted in FIG. 1 involves a probe labeled with an acceptor fluorophore. When the probe specifically interacts with a target strand, thus producing a double stranded region of DNA, the free donor intercalator intercalates. Some of the non-sequence specific intercalated donor fluorophore is positioned in proximity to the acceptor. It has been discovered according to the invention that the FRET signal increases many fold (for example 10³) after the donor is intercalated (over that of the interaction between free donor intercalator and acceptor in the solution). Thus, when the probe is bound, the energy transferred between the donor and acceptor fluorophores is greater. It will be apparent that binding of the probe can be detected either by the reduction (or elimination) of emission signal from the intercalator fluorophore or the production of emission signal from the probe fluorophore, assuming that the intercalator fluorophore is the donor and the probe fluorophore is the acceptor. D refers to an intercalator that functions as a donor and A refers to an acceptor.

FIG. 2 is a schematic diagram of another example of the methods of the invention. In FIG. 2 the intercalator fluorophore and the probe fluorophore are both tethered to the probe. In the example the intercalator fluorophore is tethered via a flexible linker, such that the intercalator fluorophore is capable of intercalating into the double stranded DNA once the probe binds to the target.

Another example of the methods of the invention which is not depicted specifically in the Figures involves the use of two probes, one tethered to the intercalator fluorophore and the other tethered to the probe fluorophore. In this embodiment of the invention the two probes are capable of hybridizing to adjacent sections of the nucleic acid. Preferably both probes are sequence-specific, but one or both may be non-sequence-specific. The term “adjacent sections of the nucleic acid” as used herein refers to two sections along the length of a nucleic acid which are in close proximity to one another in the primary structure of the nucleic acid. Two probes may hybridize to adjacent sections of the nucleic acid by hybridizing to immediately adjacent sections or to spaced adjacent sections. The term “immediately adjacent sections” refers to two sections of a nucleic acid which have no intervening units, i.e., two sections of a nucleic acid that are directly connected to one another without any intervening nucleotides. The term “spaced adjacent sections” refers to two sections of a nucleic acid that are separated from one another by one or more units, i.e., two sections of a nucleic acid that are connected to one another by one or more intervening nucleotides.

It is to be understood that sequence information is derived from the hybridization of the sequence specific probe(s) to the nucleic acid target. Hybridization of the sequence specific probe and its location along the length of the nucleic acid target is indicated by FRET. FRET can be detected in at least one of two ways: fluorescence or quenching. In fluorescence, a detector is set to the emission spectra of the acceptor fluorophore and binding of the sequence specific probe is indicated by energy transfer from the donor to the acceptor and fluorescence from the acceptor. In quenching, the detector is set to the emission spectra of the donor fluorophore and binding of the sequence specific probe is indicated by energy transfer from the donor to the acceptor and quenching of emission from the donor. In either mode, fluorescence from the intercalator fluorophore is increased upon actual intercalation. In addition, intercalators prefer binding to double stranded nucleic acids rather than single stranded nucleic acids. Therefore, once the sequence specific probe is bound, emission and/or energy transfer from the donor fluorophore will increase. It will be understood that minor variations of the foregoing will apply in the various aspects of the invention.

The fluorophores may be directly or indirectly tethered to an internal unit, a terminal unit, or a combination of internal and terminal units on the probe. The fluorophores may both be directly linked to the nucleic acid or indirectly linked to the nucleic acid through the use of one or more linkers. The fluorophores may be both tethered to individual internal or terminal nucleotides or one may be tethered to an internal nucleotide and one may be tethered to a terminal nucleotide. The term “terminal unit” or “terminal nucleotide” refers to an end unit or nucleotide on the probe, i.e., a 5′ or 3′ end. The term “internal unit” or “internal nucleotide” refers to a unit or nucleotide that is positioned between the end units or nucleotides of the probe.

It may be desirable, in some instances, to tether either of the fluorophores to the probe via a spacer or linker molecule. Preferably, the linker is a length within an optimal range to allow the fluorophore to interact with its complementary fluorophore.

These spacers can be any of a variety of molecules, preferably non-active, such as nucleotides or multiple nucleotides, straight or branched saturated or unsaturated carbon chains of carbon, phospholipids, and the like, whether naturally occurring or synthetic. Additional spacers include alkyl and alkenyl carbonates, carbamates, and carbamides.

A wide variety of spacers can be used, many of which are commercially available, for example, from sources such as Boston Probes, Inc. (now Applied Biosystems, Inc.). Spacers are not limited to organic spacers, and rather can be inorganic also (e.g., —O—Si—O—, or O—P—O—). Additionally, they can be heterogeneous in nature (e.g., composed of organic and inorganic elements). Essentially any molecule having the appropriate size restrictions and capable of being linked to a fluorophore and probe can be used as a spacer.

In some embodiments the linker is one or more nucleotides. The use of nucleotide(s) as a linker is particularly useful when the probes are nucleic acid, PNA or LNA probes, because of the ease of producing the probe-linker construct. In some embodiments the linker comprises or consists solely of thymidine (T) nucleotides.

The methods of the invention can be used to generate unit specific information about a nucleic acid by capturing signals arising from the labeled nucleic acid using the devices described herein and elsewhere to manipulate the nucleic acid. As used herein the term “unit specific information” refers to any structural information about one, some, or all of the units of the nucleic acid. The structural information obtained by analyzing a nucleic acid may include the identification of characteristic properties of the nucleic acid which (in turn) allows, for example, for the identification of the presence of a nucleic acid in a sample, determination of the relatedness of nucleic acids, identification of the size of the nucleic acid, identification of the proximity or distance between two or more individual units or unit specific markers of a nucleic acid, identification of the order of two or more individual units or unit specific markers within a nucleic acid, and/or identification of the general composition of the units or unit specific markers of the nucleic acid. Since the structure and function of biological molecules are interdependent, the structural information can reveal important information about the function of the nucleic acid.

Thus, the term “analyzing a nucleic acid” as used herein means obtaining some information about the structure of the nucleic acid such as its size, the order of its units, its relatedness to other nucleic acids, the identity of its units, or its presence or absence in a sample.

The term “nucleic acid” refers to multiple linked nucleotides (i.e., molecules comprising a sugar (e.g., ribose or deoxyribose) linked to an exchangeable organic base, which is either a pyrimidine (e.g., cytosine (C), thymidine (T) or uracil (U)) or a purine (e.g., adenine (A) or guanine (G)). “Nucleic acid” and “nucleic acid molecule” are used interchangeably and refer to oligoribonucleotides as well as oligodeoxyribonucleotides. The terms shall also include polynucleosides (i.e., a polynucleotide minus a phosphate) and any other organic base containing nucleic acid. The nucleic acid being analyzed and/or labeled is referred to as the nucleic acid target.

Nucleic acid targets and nucleic acid probes may be DNA or RNA, although they are not so limited. DNA may be genomic DNA such as nuclear DNA or mitochondrial DNA. RNA may be mRNA, mRNA, rRNA and the like. Nucleic acids may be naturally occurring such as those recited above, or may be synthetic such as cDNA.

Harvest and isolation of nucleic acids are routinely performed in the art and suitable methods can be found in standard molecular biology textbooks. The nucleic acid may be harvested from a biological sample such as a tissue or a biological fluid. The term “tissue” as used herein refers to both localized and disseminated cell populations including. but not limited, to brain, heart, breast, colon, bladder, uterus, prostate, stomach, testis, ovary, pancreas, pituitary gland, adrenal gland, thyroid gland, salivary gland, mammary gland, kidney, liver, intestine, spleen, thymus, bone marrow, trachea, and lung. Biological fluids include saliva, sperm, serum, plasma, blood and urine, but are not so limited. Both invasive and non-invasive techniques can be used to obtain such samples and are well documented in the art.

The methods of the invention may be performed in the absence of prior nucleic acid amplification in vitro. In some preferred embodiments, the nucleic acid is directly harvested and isolated from a biological sample (such as a tissue or a cell culture), without its amplification. Accordingly, some embodiments of the invention involve analysis of “non in vitro amplified nucleic acids”. As used herein, a “non in vitro amplified nucleic acid” refers to a nucleic acid that has not been amplified in vitro using techniques such as polymerase chain reaction or recombinant DNA methods.

A non in vitro amplified nucleic acid may, however, be a nucleic acid that is amplified in vivo (e.g., in the biological sample from which it was harvested) as a natural consequence of the development of the cells in the biological sample. This means that the non in vitro nucleic acid may be one which is amplified in vivo as part of gene amplification, which is commonly observed in some cell types as a result of mutation or cancer development.

In some embodiments, the invention embraces nucleic acid derivatives as targets and/or probes. As used herein, a “nucleic acid derivative” is a non-naturally occurring nucleic acid. Nucleic acid derivatives may contain non-naturally occurring elements such as non-naturally occurring nucleotides and non-naturally occurring backbone linkages. These include substituted purines and pyrimidines such as C-5 propyne modified bases, 5-methylcytosine, 2-aminopurine, 2-amino-6-chloropurine, 2,6-diaminopurine, hypoxanthine, 2-thiouracil and pseudoisocytosine. Other such modifications are well known to those of skill in the art.

The nucleic acids may also encompass substitutions or modifications, such as in the bases and/or sugars. For example, they include nucleic acids having backbone sugars which are covalently attached to low molecular weight organic groups other than a hydroxyl group at the 3′ position and other than a phosphate group at the 5′ position. Thus, modified nucleic acids may include a 2′-O-alkylated ribose group. In addition, modified nucleic acids may include sugars such as arabinose instead of ribose.

The nucleic acids may be heterogeneous in backbone composition thereby containing any possible combination of nucleic acid units linked together such as peptide nucleic acids (which have amino acid linkages with nucleic acid bases, and which are discussed in greater detail herein). In some embodiments, the nucleic acids are homogeneous in backbone composition.

As used herein with respect to linked units of a nucleic acid, “linked” or “linkage” means two entities bound to one another by any physicochemical means. Any linkage known to those of ordinary skill in the art, covalent or non-covalent, is embraced. Natural linkages, which are those ordinarily found in nature connecting the individual units of a particular nucleic acid, are most common. Natural linkages include, for instance, amide, ester and thioester linkages. The individual units of a nucleic acid analyzed by the methods of the invention may be linked, however, by synthetic or modified linkages. Nucleic acids where the units are linked by covalent bonds will be most common but those that include hydrogen bonded units are also embraced by the invention. It is to be understood that all possibilities regarding nucleic acids appear equally to nucleic acid targets and nucleic acid probes.

The nucleic acids are analyzed with fluorophore pairs. A fluorophore or fluorescent label is a substance which is capable of exhibiting fluorescence within a detectable range. Fluorophores include, but are not limited to, fluorescein, isothiocyanate, fluorescein amine, eosin, rhodamine, dansyl, umbelliferone, 5-carboxyfluorescein (FAM), 2‘7’-dimethoxy-4‘5’-dichloro-6-carboxyfluorescein (JOE), rhodamine, 6 carboxyrhodamine (R6G), N,N,N′,N′-tetramethyl-6-carboxyrhodamine (TAMRA), 6-carboxy-X-rhodamine (ROX), 4-(4′-dimethylaminophenylazo) benzoic acid (DABCYL), 5-(2′-aminoethyl) aminonaphthalene-1-sulfonic acid (EDANS), 4-acetamido-4′-isothiocyanatostilbene-2, 2′disulfonic acid, acridine, acridine isothiocyanate, r-amino-N->3-vinylsulfonyl)phenyl!naphthalimide-3,5, disulfonate (Lucifer Yellow VS), N-(4-anilino-1-naphthyl)maleimide, anthranilamide, Brilliant Yellow, coumarin, 7-amino-4-methylcoumarin, 7-amino-4-trifluoromethylcouluarin (Coumaran 151), cyanosine, 4′,6-diaminidino-2-phenylindole (DAPI), 5′,5″-diaminidino-2-phenylindole (DAPI), 5′,5″-dibromopyrogallol-sulfonephthalein (Bromopyrogallol Red), 7-diethylamino-3-(4′-isothiocyanatophenyl)-4-methylcoumarin diethylenetriamine pentaacetate, 4,4′-diisothiocyanatodihydro-stilbene-2,2′-disulfonic acid, 4,4′-diisothiocyanatostilbene-2,2′-disulfonic acid, 4-dimethylaminophenylazophenyl-4′-isothiocyanate (DABITC), eosin isothiocyanate, erythrosin B, erythrosin isothiocyanate, ethidium, 5-(4,6-dichlorotriazin-2-yl) aminofluorescein (DTAF), QFITC (XRITC), fluorescamine, IR144, IR1446, Malachite Green isothiocyanate, 4-methylumbelliferone, ortho cresolphthalein, nitrotyrosine, pararosaniline, Phenol Red, B-phycoerythrin, o-phthaldialdehyde, pyrene, pyrene butyrate, succinimidyl 1-pyrene butyrate, Reactive Red 4 (Cibacron. RTM. Brilliant Red 3B-A), lissamine rhodamine B sulfonyl chloride, rhodamine B, rhodamine 123, rhodamine X isothiocyanate, sulforhodamine B, sulforhodamine 101, sulfonyl chloride derivative of sulforhodamine 101, (Texas Red), tetramethyl rhodamine, tetramethyl rhodamine isothiocyanate (TRITC), riboflavin, rosolic acid, and terbium chelate derivatives.

Fluorophore pairs are two fluorophores that are capable of undergoing FRET to produce or eliminate a detectable signal when positioned in proximity to one another. Examples of donors include Ha10TAlexa488, Ha10TAlexa546, Ha10TBODIPY493, Ha10TOyster556, Hal OTFluor (FAM), Ha10TCy3, and HA10TTR (Tamra). Examples of acceptors include HACy5, HaAlexa594, HAAlexa647, and HaOyster656.

An intercalator fluorophore is a fluorophore that is capable of non-sequence specific binding to preferably double stranded nucleic acids. The intercalators include compounds such as phenanthridines and acridines (e.g., ethidium bromide, propidium iodide, hexidium iodide, dihydroethidium, ethidium homodimer-1 and -2, ethidium monoazide, and ACMA) and acridine orange. All of the aforementioned intercalators are commercially available from suppliers such as Molecular Probes, Inc. The invention can also be practiced using other non-sequence specific binding agents such as minor groove binding agents. Minor groove binding agents are compounds that bind to the minor groove of preferably a double stranded nucleic acid helix in a relatively non-sequence specific manner. Examples include indoles and imidazoles (e.g., Hoechst 33258, Hoechst 33342, Hoechst 34580 and DAPI). Minor groove binding agents can be used in place of intercalator or probe fluorophores, for example.

Fluorescence may be measured using a fluorometer. The optical emission from the fluorescence molecule, whether the acceptor or the donor, can be detected by the fluorometer and processed as a signal. When fluorescence is being measured in a sample fixed to various portions of a surface (e.g., when the nucleic acid is fixed), the surface can be moved using a multi-access translation stage in order to position the different areas of the surface, such that the signal can be collected. When the fluorescence is measured in solution other methods can be used for detecting the signal including the linear analysis methods described herein. Many types of fluorometers have been developed. For instance, an example of an instrument for measuring FRET is described in U.S. Pat. No. 5,911,952.

The nucleic acid is labeled with one or more sequence specific probes. “Sequence specific” when used in the context of a nucleic acid probe means that the probe recognizes a particular linear arrangement of nucleotides or derivatives thereof. In preferred embodiments, the linear arrangement includes contiguous nucleotides or derivatives thereof that each bind to a corresponding complementary nucleotide on the nucleic acid target. In some embodiments, however, the sequence may not be contiguous as there may be one, two, or more nucleotides that do not have corresponding complementary residues on the target.

It is to be understood that any nucleic acid analog that is capable of recognizing a nucleic acid molecule with structural or sequence specificity can be used as a nucleic acid probe. In most instances, the nucleic acid probes will form at least a Watson-Crick bond with the nucleic acid target. In other instances, the nucleic acid probe can form a Hoogsteen bond with the nucleic acid target, thereby forming a triplex. A nucleic acid sequence that binds by Hoogsteen binding enters the major groove of a nucleic acid target and hybridizes with the bases located there. Examples of these latter probes include molecules that recognize and bind to the minor and major grooves of nucleic acids (e.g., some forms of antibiotics). In some embodiments, the nucleic acid probes can form both Watson-Crick and Hoogsteen bonds with the nucleic acid target. Bis PNA probes, for instance, are capable of both Watson-Crick and Hoogsteen binding to a nucleic acid.

In some embodiments, the nucleic acid probe is a peptide nucleic acid (PNA), a bis PNA clamp, a pseudocomplementary PNA, a locked nucleic acid (LNA), DNA, RNA, or co-nucleic acids of the above such as DNA-LNA co-nucleic acids. In some instances, the nucleic acid target can also be comprised of any of these elements.

PNAs are DNA analogs having their phosphate backbone replaced with 2-aminoethyl glycine residues linked to nucleotide bases through glycine amino nitrogen and methylenecarbonyl linkers. PNAs can bind to both DNA and RNA targets by Watson-Crick base pairing, and in so doing form stronger hybrids than would be possible with DNA or RNA based probes.

PNAs are synthesized from monomers connected by a peptide bond (Nielsen, P. E. et al. Peptide Nucleic Acids Protocols and Applications, Norfolk: Horizon Scientific Press, p. 1-19 (1999)). They can be built with standard solid phase peptide synthesis technology. PNA chemistry and synthesis allows for inclusion of amino acids and polypeptide sequences in the PNA design. For example, lysine residues can be used to introduce positive charges in the PNA backbone. All chemical approaches available for the modifications of amino acid side chains are directly applicable to PNAs.

PNA has a charge-neutral backbone, and this attribute leads to fast hybridization rates of PNA to DNA (Nielsen, P. E. et al. Peptide Nucleic Acids, Protocols and Applications, Norfolk: Horizon Scientific Press, p. 1-19 (1999)). The hybridization rate can be further increased by introducing positive charges in the PNA structure, such as in the PNA backbone or by addition of amino acids with positively charged side chains (e.g., lysines). PNA can form a stable hybrid with DNA molecule. The stability of such a hybrid is essentially independent of the ionic strength of its environment (Orum, H. et al., BioTechniques 19 (3): 472-480 (1995)), most probably due to the uncharged nature of PNAs. This provides PNAs with the versatility of being used in vivo or in vitro. However, the rate of hybridization of PNAs that include positive charges is dependent on ionic strength, and thus is lower in the presence of salt.

Several types of PNA designs exist, and these include single strand PNA (ssPNA), bis PNA and pseudocomplementary PNA (pcPNA).

The structure of PNA/DNA complex depends on the particular PNA and its sequence. Single stranded PNA (ssPNA) binds to single stranded DNA (ssDNA) preferably in antiparallel orientation (i.e., with the N-terminus of the ssPNA aligned with the 3′ terminus of the ssDNA) and with a Watson-Crick pairing. PNA also can bind to DNA with a Hoogsteen base pairing, and thereby forms triplexes with double stranded DNA (dsDNA) (Wittung, P. et al., Biochemistry 36: 7973 (1997)).

Single strand PNA is the simplest of the PNA molecules. This PNA form interacts with nucleic acids to form a hybrid duplex via Watson-Crick base pairing. The duplex has different spatial structure and higher stability than dsDNA (Nielsen, P. E. et al. Peptide Nucleic Acids Protocols and Applications, Norfolk: Horizon Scientific Press, p. 1-19 (1999)). However, when different concentration ratios are used and/or in presence of complimentary DNA strand, PNA/DNA/PNA or PNA/DNA/DNA triplexes can also be formed (Wittung, P. et al., Biochemistry 36: 7973 (1997)). The formation of duplexes or triplexes additionally depends upon the sequence of the PNA. Thymine-rich homopyrimidine ssPNA forms PNA/DNA/PNA triplexes with dsDNA targets where one PNA strand is involved in Watson-Crick antiparallel pairing and the other is involved in parallel Hoogsteen pairing. Cytosine-rich homopyrimidine ssPNA preferably binds through Hoogsteen pairing to dsDNA forming a PNA/DNA/DNA triplex. If the ssPNA sequence is mixed, it invades the dsDNA target, displaces the DNA strand, and forms a Watson-Crick duplex. Polypurine ssPNA also forms triplex PNA/DNA/PNA with reversed Hoogsteen pairing.

BisPNA includes two strands connected with a flexible linker. One strand is designed to hybridize with DNA by a classic Watson-Crick pairing, and the second is designed to hybridize with a Hoogsteen pairing. The target sequence can be short (e.g., 8 bp), but the bis PNA/DNA complex is still stable as it forms a hybrid with twice as many (e.g., a 16 bp) base pairings overall. The bis PNA structure further increases specificity of their binding. As an example, binding to an 8 bp site with a probe having a single base mismatch results in a total of 14 bp rather than 16 bp.

Preferably, bis PNAs have homopyrimidine sequences, and even more preferably, cytosines are protonated to form a Hoogsteen pair to a guanosine. Therefore, bis PNA with thymines and cytosines is capable of hybridization to DNA only at pH below 6.5. The first restriction—homopyrimidine sequence only—is inherent to the mode of bis PNA binding. Pseudoisocytosine (J) can be used in the Hoogsteen strand instead of cytosine to allow its hybridization through a broad pH range (Kuhn, H., J. Mol. Biol. 286: 1337-1345 1999)).

Bis PNAs have multiple modes of binding to nucleic acids (Hansen, G. I. et al., J. Mol. Biol. 307 (1): 67-74 (2001)). One isomer includes two bis PNA molecules instead of one. It is formed at higher bis PNA concentration and has a tendency to rearrange into the complex with a single bis PNA molecule. Other isomers differ in positioning of the linker around the target DNA strands. All the identified isomers still bind to the same binding site/target.

Pseudocomplementary PNA (pcPNA) (Izvolsky, K. I. et al., Biochemistry 10908-10913 (2000)) involves two single stranded PNAs added to dsDNA. One pcPNA strand is complementary to the target sequence, while the other is complementary to the displaced DNA strand. As the PNA/DNA duplex is more stable, the displaced DNA generally does not restore the dsDNA structure. The PNA/PNA duplex is more stable than the DNA/PNA duplex and the PNA components are self-complementary because they are designed against complementary DNA sequences. Hence, the added PNAs would rather hybridize to each other. To prevent the self-hybridization of pcPNA units, modified bases are used for their synthesis including 2,6-diamiopurine (D) instead of adenine and 2-thiouracil (^{S}U) instead of thymine. While D and ^{S}U are still capable of hybridization with T and A respectively, their self-hybridization is sterically prohibited.

Locked nucleic acid (LNA) molecules form hybrids with DNA, which are at least as stable as PNA/DNA hybrids (Braasch, D. A. et al., Chem & Biol. 8 (1): 1-7 (2001)). Therefore, LNA can be used just as PNA molecules would be. LNA binding efficiency can be increased in some embodiments by adding positive charges to it. LNAs have been reported to have increased binding affinity inherently.

Commercial nucleic acid synthesizers and standard phosphoramidite chemistry are used to make LNAs. Therefore, production of mixed LNA/DNA sequences is as simple as that of mixed PNA/peptide sequences. The stabilization effect of LNA monomers is not an additive effect. The monomer influences conformation of sugar rings of neighboring deoxynucleotides shifting them to more stable configurations (Nielsen, P. E. et al. Peptide Nucleic Acids, Protocols and Applications, Norfolk: Horizon Scientific Press, p. 1-19 (1999)). Also, lesser number of LNA residues in the sequence dramatically improves accuracy of the synthesis. Naturally, most of biochemical approaches for nucleic acid conjugations are applicable to LNA/DNA constructs.

The probes can also be stabilized in part by the use of other backbone modifications. The invention intends to embrace, in addition to the peptide and locked nucleic acids discussed herein, the use of the other backbone modifications such as but not limited to phosphorothioate linkages, phosphodiester modified nucleic acids, combinations of phosphodiester and phosphorothioate nucleic acid, methylphosphonate, alkylphosphonates, phosphate esters, alkylphosphonothioates, phosphoramidates, carbamates, carbonates, phosphate triesters, acetamidates, carboxymethyl esters, methylphosphorothioate, phosphorodithioate, p-ethoxy, and combinations thereof.

Other backbone modifications, particularly those relating to PNAs, include peptide and amino acid variations and modifications. Thus, the backbone constituents of PNAs may be peptide linkages, or alternatively, they may be non-peptide linkages. Examples include acetyl caps, amino spacers such as O-linkers, amino acids such as lysine (particularly useful if positive charges are desired in the PNA), and the like. Various PNA modifications are known and probes incorporating such modifications are commercially available from sources such as Boston Probes, Inc.

One limitation of the stability of nucleic acid hybrids is the length of the probe, with longer probes leading to greater stability than shorter probes. Notwithstanding this proviso, the probes of the invention can be any length ranging from at least 4 nucleotides long to in excess of 1000 nucleotides long. In preferred embodiments, the probes are 5-100 nucleotides in length, more preferably between 5-25 nucleotides in length, and even more preferably 5-12 nucleotides in length. The length of the probe can be any length of nucleotides between and including the ranges listed herein, as if each and every length was explicitly recited herein. It should be understood that not all residues of the probe need hybridize to complementary residues in the nucleic acid target. For example, the probe may be 50 residues in length, yet only 25 of those residues hybridize to the nucleic acid target. Preferably, the residues that hybridize are contiguous with each other.

The probes are preferably single stranded, but they are not so limited. For example, when the probe is a bis PNA it can adopt a secondary structure with the nucleic acid target resulting in a triple helix conformation, with one region of the bis PNA clamp forming Hoogsteen bonds with the backbone of the target and another region of the bis PNA clamp forming Watson-Crick bonds with the nucleotide bases of the target.

The nucleic acid probe hybridizes to a complementary sequence within the nucleic acid target. The specificity of binding can be manipulated based on the hybridization conditions. For example, salt concentration and temperature can be modulated in order to vary the range of sequences recognized by the nucleic acid probes.

The polymers may be analyzed using a single molecule analysis system (e.g., a single polymer analysis system). A single molecule detection system is capable of analyzing single molecules separately from other molecules. Such a system may be capable of analyzing single molecules either in a linear manner (i.e., starting at a point and then moving progressively in one direction or another) and/or, as may be more appropriate in the present invention, in their totality. In certain embodiments in which detection is based predominately on the presence or absence of a signal, linear analysis may not be required. However, there are other embodiments embraced by the invention which would benefit from the ability to linearly analyze molecules (preferably nucleic acids) in a sample. These include applications in which the sequence of the nucleic acid is desired.

A linear polymer analysis system is a system that analyzes polymers in a linear manner (i.e., starting at one location on the polymer and then proceeding linearly in either direction therefrom). As a polymer is analyzed, the detectable labels attached to it are detected in either a sequential or simultaneous manner. When detected simultaneously, the signals usually form an image of the polymer, from which distances between labels can be determined. When detected sequentially, the signals are viewed in histogram (signal intensity vs. time), that can then be translated into a map, with knowledge of the velocity of the polymer. It is to be understood that in some embodiments, the polymer is attached to a solid support, while in others it is free flowing. In either case, the velocity of the polymer as it moves past, for example, an interaction station or a detector, will aid in determining the position of the labels, relative to each other and relative to other detectable markers that may be present on the polymer.

Accordingly, the analysis systems useful in the invention may deduce the total amount of label on a polymer, and in some instances, the location of such labels. The ability to locate and position the labels allows these patterns to be superimposed on other genetic maps, in order to orient and/or identify the regions of the genome being analyzed.

An example of a suitable system is the GeneEngine™ (U.S. Genomics, Inc., Woburn, Mass.). The Gene Engine™ system is described in PCT patent applications WO98/35012 and WO00/0975, published on Aug. 13, 1998, and Feb. 24, 2000, respectively, and in issued U.S. Pat. No. 6,355,420 B1, issued Mar. 12, 2002. The contents of these applications and patent, as well as those of other applications and patents, and references cited herein are incorporated by reference in their entirety. This system is both a single molecule analysis system and a linear polymer analysis system. It allows single nucleic acid molecules to be passed through an interaction station in a linear manner, whereby the nucleotides in the nucleic acid molecules are interrogated individually in order to determine whether there is a detectable label conjugated to the nucleic acid molecule. Interrogation involves exposing the nucleic acid molecule to an energy source such as optical radiation of a set wavelength. In response to the energy source exposure, the detectable label on the nucleotide emits a signal which is exposed to the second fluorophore of the fluorophore pair (if present in the vicinity) to produce a detectable signal. The mechanism for signal emission and detection will depend on the type of label sought to be detected.

Other single molecule nucleic acid analytical methods which involve elongation of DNA molecules can also be used in the methods of the invention. These include fiber-fluorescence in situ hybridization (fiber-FISH) (Bensimon, A. et al., Science 265 (5181): 2096-2098 (1997)). In fiber-FISH, nucleic acid molecules are elongated and fixed on a surface by molecular combing. Hybridization with fluorescently labeled probe sequences allows determination of sequence landmarks on the nucleic acid molecules. The method requires fixation of elongated molecules so that molecular lengths and/or distances between markers can be measured. Pulse field gel electrophoresis can also be used to analyze the labeled nucleic acid molecules. Pulse field gel electrophoresis is described by Schwartz, D. C. et al., Cell 37 (1): 67-75 (1984). Other nucleic acid analysis systems are described by Otobe, K. et al., Nucleic Acids Res. 29 (22): E109 (2001), Bensimon, A. et al. in U.S. Pat. No. 6,248,537, issued Jun. 19, 2001, Herrick, J. et al., Chromosome Res. 7 (6): 409: 423 (1999), Schwartz in U.S. Pat. No. 6,150,089 issued Nov. 21, 2000 and U.S. Pat. No. 6,294,136, issued Sep. 25, 2001. Other linear polymer analysis systems can also be used, and the invention is not intended to be limited to solely those listed herein.

Optical detectable signals are generated, detected and stored in a database. The signals can be analyzed to determine structural information about the nucleic acid. The signals can be analyzed by assessing the intensity of the signal to determine structural information about the nucleic acid. The computer may be the same computer used to collect data about the nucleic acids, or may be a separate computer dedicated to data analysis. A suitable computer system to implement embodiments of the present invention typically includes an output device which displays information to a user, a main unit connected to the output device and an input device which receives input from a user. The main unit generally includes a processor connected to a memory system via an interconnection mechanism. The input device and output device also are connected to the processor and memory system via the interconnection mechanism. Computer programs for data analysis of the detected signals are readily available from CCD (charge coupled device) manufacturers.

### Equivalents

The foregoing written specification is considered to be sufficient to enable one skilled in the art to practice the invention. The present invention is not to be limited in scope by examples provided, since the examples are intended as a single illustration of one aspect of the invention and other functionally equivalent embodiments are within the scope of the invention. Various modifications of the invention in addition to those shown and described herein will become apparent to those skilled in the art from the foregoing description and fall within the scope of the appended claims. The advantages and objects of the invention are not necessarily encompassed by each embodiment of the invention.

The entire contents of all of the references (including literature references, issued patents, published patent applications, and co-pending patent applications) cited throughout this application are expressly incorporated by reference herein.

1. A method for analyzing a nucleic acid comprising:
contacting a nucleic acid with an intercalator fluorophore and a sequence specific probe capable of hybridizing to the nucleic acid, wherein the probe is labeled with a probe fluorophore, and
detecting fluorescence or quenching arising from FRET between the intercalator fluorophore and the probe fluorophore to analyze the nucleic acid.

2. The method of claim 1, wherein the intercalator fluorophore is tethered to the probe.

3. The method of claim 1, wherein the intercalator fluorophore is separate from the probe.

4. The method of claim 1, wherein the intercalator fluorophore is a donor fluorophore.

5. The method of claim 1, wherein the probe fluorophore is an acceptor fluorophore.

6. The method of claim 1, wherein the probe fluorophore is tethered directly to the probe.

7. The method of claim 1, wherein the probe fluorophore is tethered to the probe through a linker.

8. The method of claim 2, wherein the intercalator fluorophore is tethered directly to the probe.

9. The method of claim 2, wherein the intercalator fluorophore is tethered to the probe through a linker.

10. The method of claim 1, wherein the probe fluorophore is tethered to a terminal nucleotide of the probe.

11. The method of claim 1, wherein the probe fluorophore is tethered to an internal nucleotide of the probe.

12. The method of claim 2, wherein the intercalator fluorophore is tethered to a terminal nucleotide of the probe.

13. The method of claim 2, wherein the intercalator fluorophore is tethered to an internal nucleotide of the probe.

14. The method of claim 1, wherein the intercalator fluorophore is tethered to a second probe which is capable of hybridizing to an adjacent section of the nucleic acid to the probe labeled with the probe fluorophore.

15. A composition comprising a probe tethered to an intercalator fluorophore and a probe fluorophore, wherein the intercalator fluorophore and the probe fluorophore comprise a fluorophore pair.

16. The composition of claim 15, wherein the intercalator fluorophore is tethered to one end of the probe and the probe fluorophore is tethered to the other end of the probe.

17. The composition of claim 15, wherein the intercalator fluorophore is a donor fluorophore.

18. The composition of claim 15, wherein the probe fluorophore is an acceptor fluorophore.

19. The composition of claim 15, wherein the probe fluorophore is tethered directly to the probe.

20. The composition of claim 15, wherein the probe fluorophore is tethered to the probe through a linker.

21. The composition of claim 15, wherein the intercalator fluorophore is tethered directly to the probe.

22. The composition of claim 15, wherein the intercalator fluorophore is tethered to the probe through a linker.
